(19) United States
(12) Patent Application Publication
(10) Pub. No.: US 2011/0172425 A1
(43) Pub. Date: Jul. 14, 2011

(54) NOVEL WATER BASED PROCESS FOR THE PREPARATION OF SUBSTITUTED DIPHENYLMETHYL PIPERAZINES

(75) Inventors: Bansi Lal, Mumbai (IN); Sanjoy Lahiri, Thane (IN); Chintamani Prabhakar Bapat, Thane (IN); Rahul Suresh Kulkarni, Thane (IN); Dilawar Kasam Mulla, Thane (IN); Ashish Yasin Hawaldar, Mumbai (IN)

(73) Assignee: CALYX CHEMICALS AND PHARMACEUTICALS PVT. LTD., Mumbai, Maharashtra (IN)

(21) Appl. No.: 13/119,346

(22) PCT Filed: Sep. 16, 2009
(86) PCT No.: PCT/IN2009/000509

§ 371 Date: Mar. 16, 2011

(30) Foreign Application Priority Data
Sep. 17, 2008 (IN) 1976/MUM/2008

(51) Int. Cl. C07D 295/088

(52) U.S. Cl. 544/396

(57) ABSTRACT

The present invention relates to a novel water based process for the preparation of substituted diphenylmethyl piperazines of Formula I and pharmaceutically acceptable salts

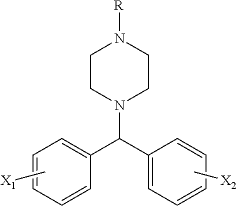

wherein X₁ and X₂ represent independently a hydrogen, a halogen, a straight or branched chain lower alkyl, alkoxy or a hydroxyl radical and R is selected from groups such as acyl, alkyl, alkenyl, aralalkyl, aralalkenyl aralkyl, and aralalkenyl or aralkenyl hydroxyalkyl, aryloxyalkyl, alkoxyalkyl, aminoalkyl or its derivative comprising, reacting a compound of Formula II, with a compound of formula R—X where R is as defined above and X is suitable leaving group which includes halides, but not limiting use of other leaving groups such as tosylate, mesylate and activated acid groups such as acyl halide, anhydrides, mixed anhydrides etc. using water as a solvent, in presence of a catalyst and a base, at 25-100° C.;

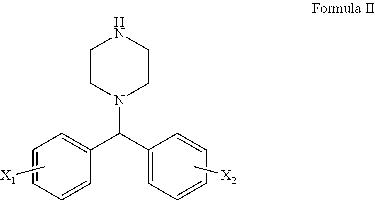

### TECHNICAL FIELD

The present invention relates to a novel water based process for the preparation of substituted diphenylmethyl piperazines of Formula I and pharmaceutically acceptable salts.

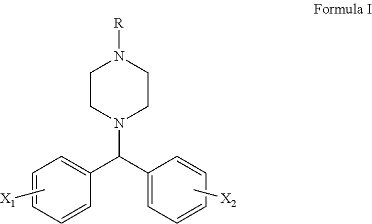

### BACKGROUND AND PRIOR ART

Substituted diphenylmethyl piperazines are known for their valuable pharmacological properties. It is well known that [bis(substituted and/or unsubstituted aryl)methyl]piperazin-1-yl compounds are used as antiasthamatics and antiallergics that inhibit leukotriene release.

U.S. Pat. No. 4,525,358 discloses (2-[2-[4-[(4-Chlorophenyl)phenylmethyl]-1-piperazinyl]ethoxyacetic acid) and its amides as antiallergic, spasmolytic and antihistamine agents.

JP 7138230 discloses 4-aralkyl-1-piperazinyl unsaturated carboxylic acid derivatives useful as antiallergic agents for the treatment of asthma and rhinitis.

WO 97/23466 describes the preparation of N-diarylmethylpiperazines as analgesics.

U.S. Pat. No. 6,451,801 explains the dual activity of these compounds as possessing both lipoxygenase inhibition properties as well as antihistaminergic properties. Piperazine moieties with lipophilic substituents are often present in cardiovascular drugs also. For example, 2-(4-diphenylmethyl-1-piperazinyl)ethyl methyl 1,4-dihydro-2,6-dimethyl-4-(3-nitrophenyl)-3,5-pyridinedicarboxylate dihydrochloride has been selected as a potent and long-acting antihypertensive drug from a series of analogues with piperazinylalkyl ester side chains. Detailed description on the activity as well as the chemistry of some of the molecules containing diphenylmethyl piperazine is cited below.

Cetirizine of Formula Ia (2-[2-[4-[(4-Chlorophenyl)phenylmethyl]-1-piperazinyl]ethoxyacetic acid) and its dihydrochloride salt are well established as drugs for the treatment of allergic syndromes, such as chronic and allergic rhinitis, allergic conjunctivitis and urticaria.

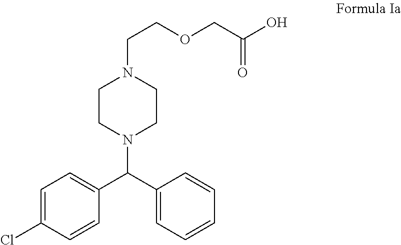

Meclizine of Formula Ib (1-[(4-chlorophenyl)-phenyl-methyl]-4-[(3-methylphenyl)methyl]piperazine) is an antihistamine considered to be an antiemetic and is most commonly used to inhibit nausea and vomiting.

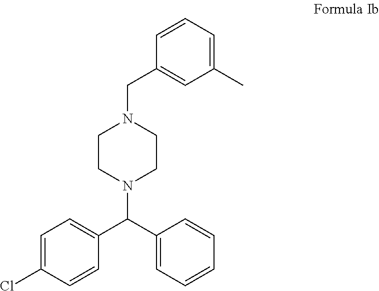

Hydroxyzine of Formula Ic (2-(2-{4-[(4-chlorophenyl)(phenyl)methyl]piperazin-1-yl}ethoxy)ethanol) is a first-generation antihistamine, of the piperazine class that is an H₁ receptor antagonist. It is used primarily for the treatment of itches and irritations, an antiemetic for the reduction of nausea, as a weak analgesic by itself and as an opioid potentiator, and as an anxiolytic for the treatment of anxiety.

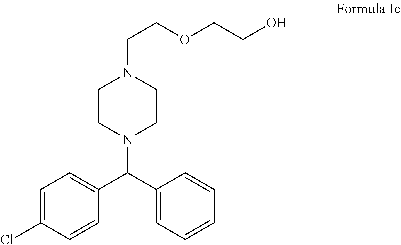

Compound of Formula Id (2-{4-[(4-chlorophenyl)(phenylmethyl]piperazin-1-yl}ethanol) is an important intermediate for the preparation of several valuable drugs such as Cetirizine of Formula Ia.

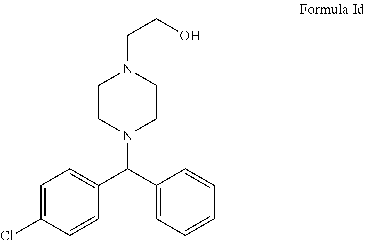

Several methods for the preparation of substituted diphenylmethyl piperazines of Formula I a dihydrochloride are known in the literature.

U.S. Pat. No. 4,525,358 describes a process for the preparation of Cetirizine of Formula Ia by reacting 1-(diphenylmethy)-piperazine of Formula III with a compound of Formula IV in an inert solvent such as benzene, toluene or xylene at reflux in presence of a base in which X is a halogen and R is OR′ or NH₂ wherein R′ is a lower alkyl radical, which is followed by hydrolysis.

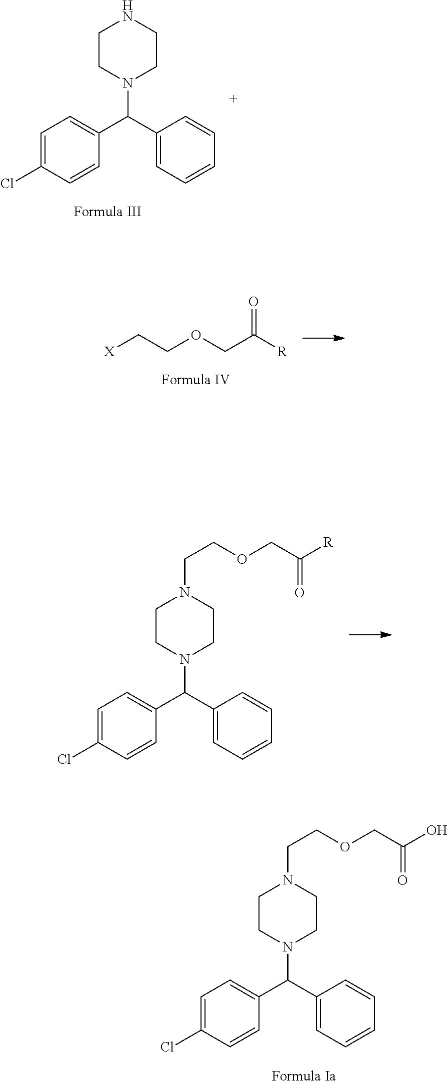

The reaction is carried out at a temperature and requires a longer reaction period of 40 hours and the resulting 2-[2-[4-[(4-Chlorophenyl)phenylmethyl]-1-piperazinyl]ethoxyacetate obtained is only 27.8% after column chromatography. GB 2225321 discloses the preparation of Cetirizine of Formula Ia by reacting 1-(diphenylmethy)-piperazine of Formula III with 2-haloethoxyacetonitrile in which X is a halogen in an inert organic solvent such as alcohol followed by the hydrolysis of the resulting nitrile using an acid or base. The reaction is carried out at a temperature of 110° C. for 11 hours and the resulting 2-[2-[4-[(4-Chlorophenyl)phenylmethyl]-1-piperazinyl]ethoxyacetonitrile is separated by column chromatography.

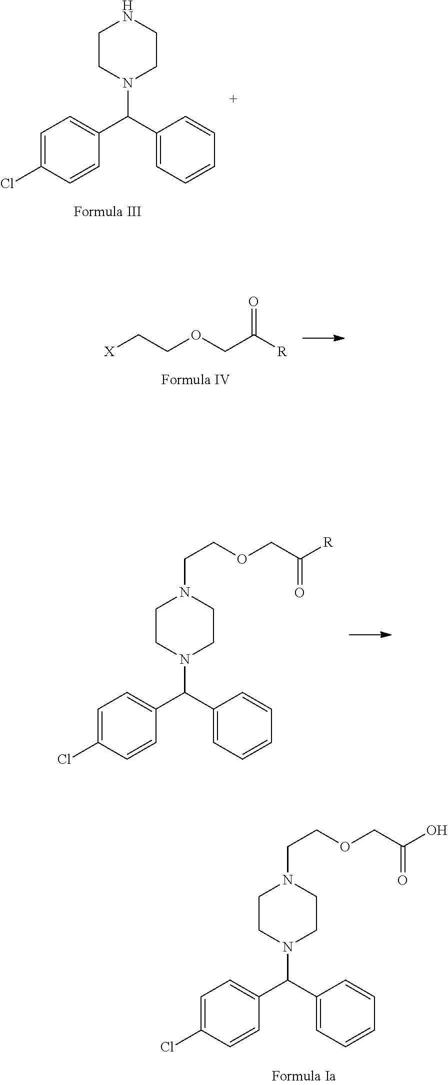

U.S. Pat. No. 6,239,277 discloses the process for the preparation of Cetirizine of Formula Ia involves the reaction of 1-(diphenylmethyl)-piperazine of Formula III and an alkoxy ester of Formula V, in which X is a leaving group and R′ is C₃ to C₁₂ branched alkyl or a cation, in an inert solvent such as dimethylformamide or 2-butanone at reflux in presence of a base.

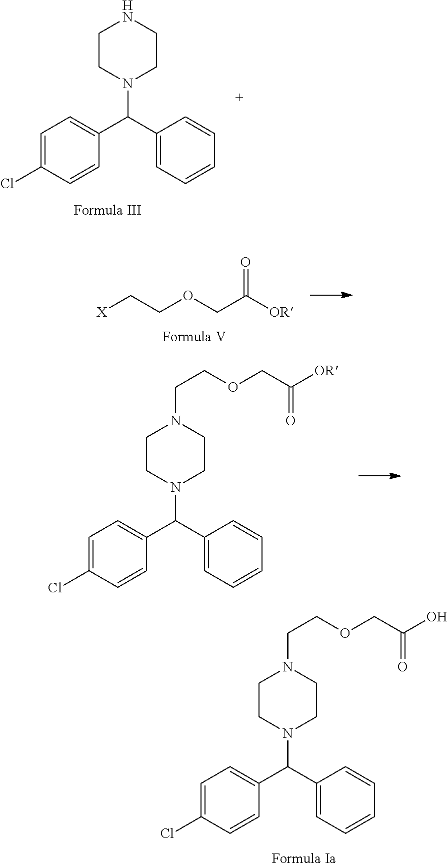

GB 2225320, GR 99100135 and WO 2004103982 disclose a process for the preparation of 2-{4-[(4-chlorophenyl)(phenylmethyl]piperazin-1-yl}ethanol of Formula Id by reacting compound of Formula III with 2-haloethanol in which the halogen is selected from chlorine or bromine in an organic solvent such as toluene.

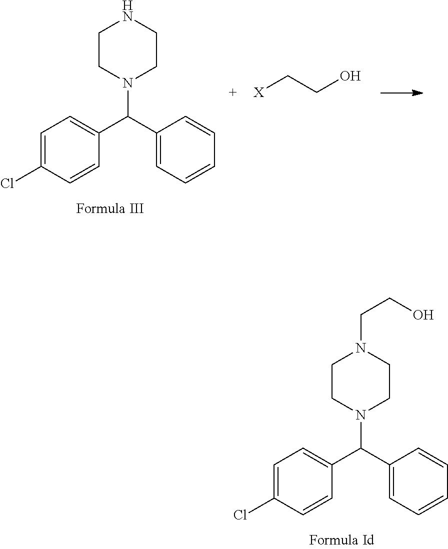

The process described in U.S. Pat. No. 2,899,436 and BE 523901 involves heating a compound of Formula III directly with Cl(CH₂)₂O(CH₂)₂OH at 150° C. for 3 hours in the absence of a solvent.

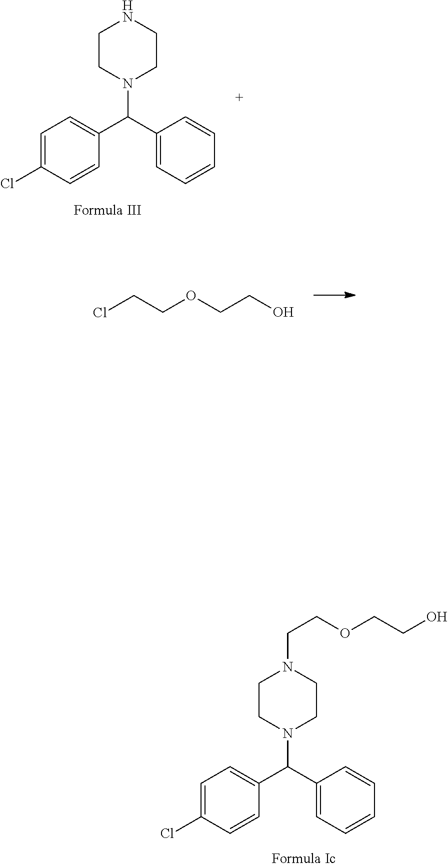

U.S. Pat. No. 2,709,169 disclose the process for the preparation of Meclizine of Formula Ib by reacting Formula III with 3-methyl benzylchloride in an organic solvent such as benzene or toluene at reflux for 3 hours.

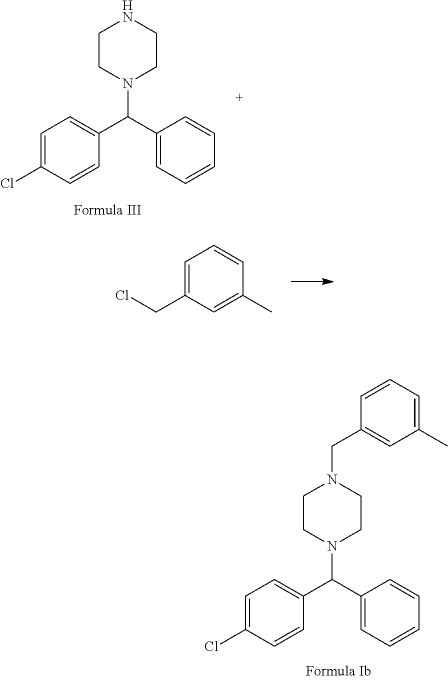

U.S. Pat. No. 6,255,487 describes a process for the preparation of racemic cetirizine of Formula Ia by reacting piperazine of Formula VI with Cl—CH₂—CH₂—O—CH₂CO₂H to get the intermediate of Formula VII followed by reacting the said intermediate with benzhydryl halide to give compound of Formula Ia. First reaction is carried out in water for 27 hours with only 12% yield and after lengthy work up procedure involving treatment with ion exchange resin followed by elution with water and evaporation of water under reduced pressure. The product is isolated by sublimation. The process is thus tedious and not commercially viable.

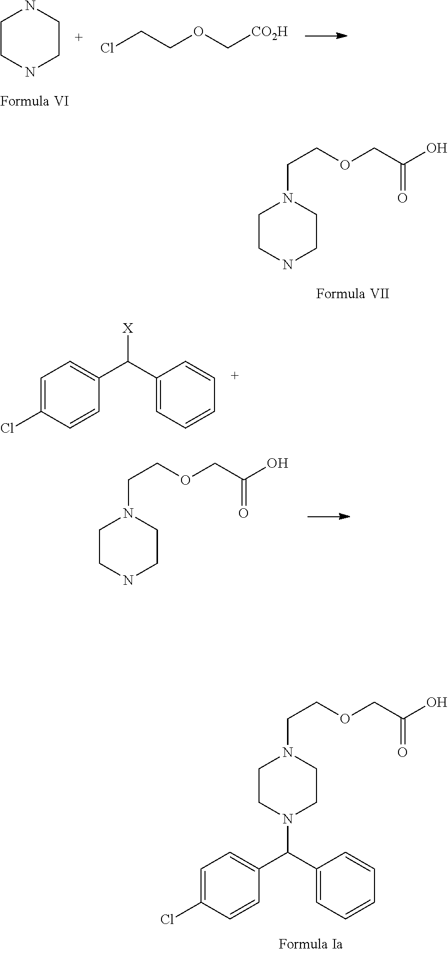

All the processes described in the prior art involve either heating the reaction mixture in an organic solvent at high temperature and/or tedious work-up and purification procedures like column chromatography which in turn leads to the use of a large amount of organic solvents making the process economically expensive. Also in most of the prior art processes, the reaction requires longer time periods and end up with lower yield of the final product. All the processes require organic solvents which are not eco-friendly.

The environmental impact and cost of solvents in drug manufacturing processes is becoming a worldwide concern. These costly, hazardous, and polluting solvents are then disposed of through environmentally unfriendly waste-disposal processes, often contributing to global warming, substantial energy consumption, ground water contamination etc.

In view of the adverse impacts of organic solvents to the environment and human health, there is a continuous need to develop an environment friendly and economical process for the preparation of substituted diphenylmethyl piperazines of Formula I in high yield and purity.

The inventors of the present invention have surprisingly found out an environment friendly and cost effective process for the preparation of substituted diphenylmethyl piperazines of Formula I from compound of Formula II using water as a solvent in presence of a catalyst and a base.

### OBJECTS OF THE INVENTION

It is an object of the present invention to provide a novel process for the preparation of substituted diphenylmethyl piperazines of Formula I and pharmaceutically acceptable salts thereof.

It is another object of the present invention to provide a water based process without using organic solvent for the preparation of substituted piperazines of Formula I from a compound of Formula II.

It is another object of the present invention to provide an environment friendly process for the preparation of substituted diphenylmethyl piperazines of Formula I and pharmaceutically acceptable salts thereof by avoiding the use of hazardous organic solvents.

It is yet another object of the present invention to provide a cost effective process for the preparation of substituted diphenylmethyl piperazines of Formula I and pharmaceutically acceptable salts thereof in good yield and with high purity.

It is further object of the present invention to provide a commercially viable process for the production of substituted diphenylmethyl piperazines of Formula I and pharmaceutically acceptable salts.

### SUMMARY OF THE INVENTION

According to an aspect of the present invention there is provided a novel process for the preparation of substituted diphenylmethyl piperazines of Formula I and pharmaceutically acceptable salts thereof

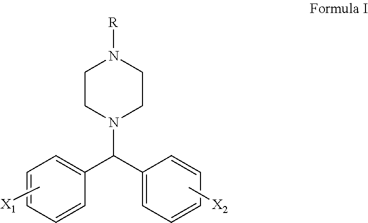

wherein X₁ and X₂ represent independently a hydrogen, a halogen, a straight or branched chain lower alkyl, alkoxy or a hydroxyl radical and R is selected from groups such as acyl, alkyl, alkenyl, aralalkyl, aralalkenyl, hydroxyalkyl, aryloxyalkyl, alkoxyalkyl, aminoalkyl or its derivatives comprising the steps:
(a) reacting a compound of Formula II with a compound of Formula R—X where R is as defined above and X is suitable leaving group which includes halides, but not limiting use of other leaving groups such as tosylate, mesylate and activated acid groups such as acyl halide, anhydrides, mixed anhydrides etc. using water as a solvent in presence of a catalyst and a base at 25-100° C.;

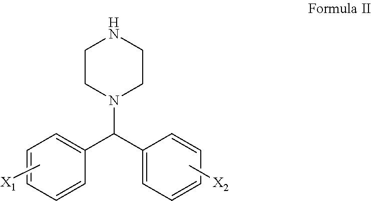

(b) continuing the reaction at 25-100° C. for 0.5-10 hours;
(c) isolating the product either by separation or by extraction using an organic solvent;
(d) optionally converting the product of Formula I to its pharmaceutically acceptable salt.

### DETAILED DESCRIPTION OF THE INVENTION

The present invention relates to a novel water based process for the preparation of substituted diphenylmethyl piperazines of Formula I and pharmaceutically acceptable salts thereof.

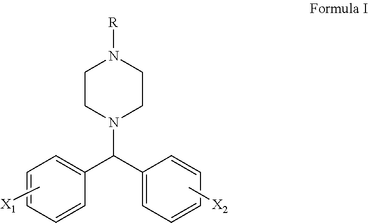

wherein X₁ and X₂ represent independently a hydrogen, a halogen, a straight or branched chain lower alkyl, alkoxy or a hydroxyl radical and R is selected from groups such as acyl, alkyl, alkenyl, aralalkyl, aralalkenyl aralkyl, and aralalkenyl or aralkenyl hydroxyalkyl, aryloxyalkyl, alkoxyalkyl, aminoalkyl or its derivative comprising,
(a) reacting a compound of Formula II, with a compound of Formula R—X where R is as defined above and X is suitable leaving group which includes halides, but not limiting use of other leaving groups such as tosylate, mesylate and activated acid groups such as acyl halide, anhydrides, mixed anhydrides etc. using water as a solvent, in presence of a catalyst and a base, at 25-100° C.;

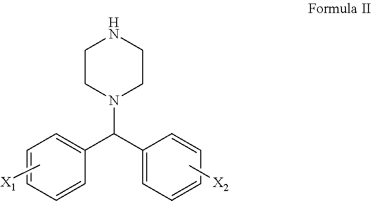

(b) continuing the reaction at 25-100° C. for 0.5-10 hours;
(c) isolating the product either by separation or by extraction using an organic solvent;
(d) optionally converting the product of Formula I to its pharmaceutically acceptable salts;

In particular, the present invention relates to a novel process for the preparation of substituted diphenylmethyl piperazines of Formula I by reacting a compound of Formula II wherein X₁ and X₂ are as defined above, with R—X wherein R and X are as defined above using water as solvent, in presence of a catalyst and a base, at a temperature of 25-100° C. for 0.5-10 hours. The reaction is shown in Scheme I.

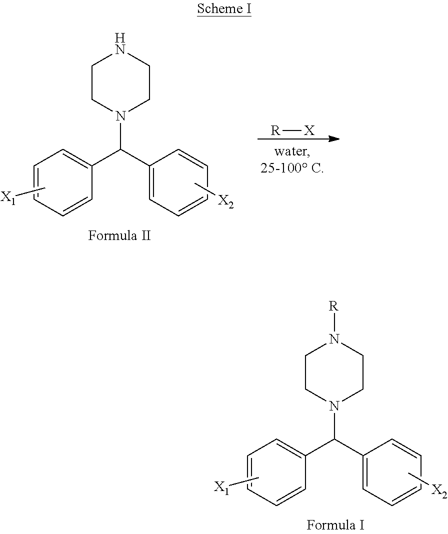

The compound of Formula II is prepared by well known prior art processes.

The term ‘aralalkyl’ denotes linear or branched alkyl radicals containing substituted or unsubstituted aryl group, the term ‘aralalkenyl’ denotes linear or branched alkenyl radicals containing substituted or unsubstituted aryl group, the term ‘hydroxyalkyl’ denotes linear or branched alkyl radical substituted with one or more hydroxyl groups, the term ‘aryloxyalkyl’ denotes alkyl radical containing substituted or unsubstituted aryloxy groups wherein the substituents include groups such as —OH, —OR, —COOH, CONH2, —CONHR, —CONR₂, —COOR—NH₂, —NHR, NR₂, —OCOR etc., the term ‘alkoxyalkyl’ means alkyl radical containing substituted or unsubstituted alkoxy groups wherein the substituents include groups such as —OH, —OR, —COOH, —CONH2, —CONHR, —CONR₂, —COOR—NH₂, —NHR, NR₂, —OCOR etc, the term ‘aminoalkyl’ refers to alkyl radical containing monosubstituted, disubstituted or unsubstituted amino groups.

Especially, preferred compounds of Formula I include,
1. (±)-[2-[4-[(4-Chlorophenyl)phenylmethyl]-1-piperazinyl]ethoxy]acetic acid dihydrochloride
2. 2-[2-[4-[(4-Chlorophenyl)phenylmethyl]-1-piperazinyl]ethoxy]ethanol
3. 1-[(4-Chlorophenyl)phenylmethyl]-4-[(3-methylphenyl)methyl]piperazine
4. 1-[(4-Chlorophenyl)phenylmethyl]-4-[[4-(1,1-dimethylethyl)phenyl]methyl]piperazine
5. 1-[(4-Chlorophenyl)phenylmethyl]-4-methylpiperazine
6. 1-(Diphenylmethyl)-4-(3-phenyl-2-propenyl)piperazine
7. 1-Diphenyl methyl-4-methylpiperazine
8. 1-(Diphenylmethyl)-4-[3-(2-phenyl-1,3-dioxolan-2-yl)propyl]piperazine
9. 2-[2-[2-[4-[(4-Chlorophenyl)phenylmethyl]-1-piperazinyl]ethoxy]ethoxy]ethanol
10. 1-[(4-Chlorophenyl)phenylmethyl]-4-(3-phenyl-2-propenyl)piperazine
11. 1-[3-[4-(Diphenylmethyl)-1-piperazinyl]propyl]-1,3-dihydro-2H-benzimidazol-2-one
12. 1,4-Dihydro-2,6-dimethyl-4-(3-nitrophenyl)-3,5-pyridinedicarboxylic acid 2-[4-(diphenylmethyl)-1-piperazinyl]ethyl methyl ester
13. 1-[Bis(4-fluorophenyl)methyl]-4-[(2,3,4-trimethoxyphenyl)methyl]piperazine
14. (E)-1-[Bis(4-fluorophenyl)methyl]-4-(3-phenyl-2-propenyl)piperazine
15. 4-[3-[4-(Diphenylmethyl)piperazin-1-yl]-2-hydroxypropoxy]-1H-indole-2-carbonitrile
16. 2-[2-[4-(4-Chlorobenzhydryl)piperazino]ethoxy]ethyl 2-(3-benzoylphenyl)propionate dimaleate
17. (±)-1-(3,4-Dimethoxyphenyl)-2-(4-diphenylmethyl-1-piperazinyl)ethanol dihydrochloride
18. 2,6-Dimethyl-3-nitro-4-[2-(4-diphenylmethyl-1-piperazinyl)ethylamino]pyridine
19. 2-[4-[4-(diphenylmethyl)piperazin-1-yl]phenyl]ethyl methyl diester dihydrochloride
20. 2-[2-[4-[Bis(4-fluorophenyl)methyl]piperazin-1-yl]ethoxy]acetic acid
21. 4-[4-[4-(Diphenylmethyl)piperazin-1-yl]butoxy]-2,3,6-trimethylphenol dihydrochloride
22. 4-Benzyl-1-(4-tert-butyl-3′-hydroxybenzhydryl)piperazine dihydrochloride
23. 4-(Diphenylmethyl)-1-[3-phenylprop-2(E)-enyl]-1-(phosphonooxymethyl)piperazin-1-ium trifluoroacetate
24. N-[4-[4-[4-[4-[Bis(4-fluorophenyl)methyl]piperazin-1-yl]butoxy]phenyl]-3-butynyl]-N-hydroxyurea and
25. 1-Allyl-4-(diphenylmethyl)piperazine dihydrochloride.

Detailed description of the process for some of these molecules is given below.

In an aspect of the present invention there is provided a cost effective process for the preparation of the Cetirizine of Formula Ia and pharmaceutically acceptable salts thereof such as Cetirizine dihydrochloride

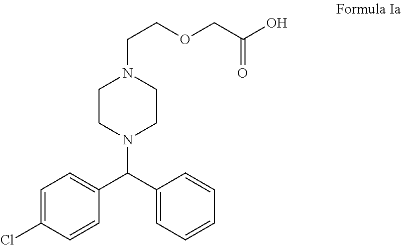

by reacting a compound of Formula III with Cl(CH₂)₂OCH₂CONH₂ using water as a solvent in presence of a catalyst and a base at a temperature of 25-100° C. for 0.5-10 hours followed by hydrolysis using-known methods

In another aspect of the present invention there is provided a cost effective process for the preparation of the Meclizine of Formula Ib and pharmaceutically acceptable salts thereof such as Meclizine dihydrochloride

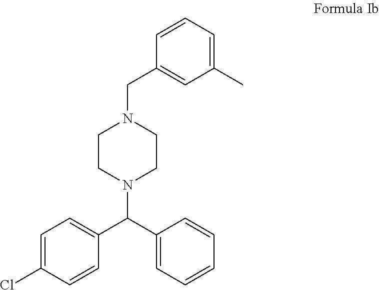

by reacting a compound of Formula III with meta ClCH₂C₆H₅CH₃ using water as a solvent in presence of a catalyst and a base at a temperature of 25-100° C. for 0.5-10 hours.

Yet another aspect of the present invention is to provide a cost effective process for the preparation of the Hydroxyzine of Formula Ic and pharmaceutically acceptable salts thereof such as Hydroxyzine dihydrochloride, pamoate etc.

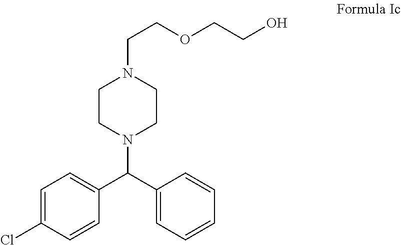

by reacting a compound of Formula III with Cl(CH₂)₂O(CH₂)₂OH using water as a solvent in presence of a catalyst and a base at a temperature of 25-100° C. for 0.5-10 hours.

Another aspect of the present invention is to provide a cost effective process for the preparation of an intermediate of Formula Id and its salts thereof

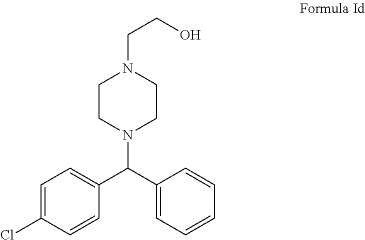

by reacting a compound of Formula III with Cl(CH₂)₂OH using water as a solvent in presence of a catalyst and a base, at a temperature of 25-100° C. for 0.5-10 hours.

In an embodiment of the present invention, the amount of water used as solvent ranges from 2 to 5 volumes, preferably from 2 to 3 volumes based on the compound of Formula II.

In an another embodiment of the present invention, the compound of Formula RX is employed in an amount ranging from 1 to 1.75 molar equivalents, preferably between 1 to 1.5 molar equivalents, more preferably between 1.1 to 1.25 molar equivalents based on the compound of Formula II.

In yet another embodiment of the present invention, the reaction is carried out at temperature between 25 to 100° C., preferably between 30 to 90° C., more preferably between 60 to 80° C.

In yet another embodiment of the present invention the reaction time varies from 0.5 to 10 hours, preferably between 1 to 7 hours, more preferably between 2 to 5 hours.

In one more embodiment of the present invention, the catalyst used is selected from a phase transfer catalyst or an alkali metal halide. Suitable phase transfer catalyst used herein include, but are not limited to, quaternary ‘onium’ salt of nitrogen or phosphorous, substituted with a residue such as alkyl or aralalkyl group, preferably tetraalkylammonium halide or trialkylaryl ammonium halide. The preferred alkali metal halide is potassium iodide.

In yet another embodiment of the present invention, the catalyst used is in an amount ranging from 0.1 to 1 wt % based on the compound of Formula II and preferably between 0.1 to 0.5 wt %; more preferably between 0.25 to 0.5 wt %.

In another embodiment of the present invention, the base used is selected from inorganic or organic bases. The inorganic base is selected from alkali metal carbonate, bicarbonate or alkaline earth metal carbonate, bicarbonate, such as potassium carbonate or sodium carbonate, and the preferred organic base is triethylamine.

In yet another embodiment of the present invention, the solvent used for extraction is selected from aromatic hydrocarbons, ethers, esters, halogenated hydrocarbons or alcohols.

The present invention is illustrated below by way of examples. Details of the invention provided in the following examples are given by the way of illustration only and should not be construed to limit the scope of the present invention.

### Example I

### (±)2-(2-{4-[(4-chlorophenyl)(phenyl)methyl]piperazin-1-yl}ethoxy)ethanol dihydrochloride (Hydroxyzine dihydrochloride)

N-(4-Chloro benzhydril) piperazine (100 gm, 0.35 mol) was taken in water (150 ml) and stirred at 25° C. Potassium carbonate (96.6 gm, 0.7 mol), and tetrabutyl ammonium bromide (0.5 g) were added in sequence into the reaction mixture while stirring it. 2-(2-chloroethoxy)ethanol (64.9 gm, 0.52 mol) dissolved in water (150 ml) was then added into the reaction mixture. The reaction mixture was heated while stirring at 80° C. for 5 h. It was cooled to room temperature and extracted with ethyl acetate (100 ml). The ethyl acetate layer was washed with water. The ethyl acetate layer was concentrated to obtain the hydroxyzine free base (128.0 g, Yield 98%, purity by HPLC: 99%), which was converted to its dihydrochloride salt by usual procedure.

IR (neat): 3356, 2285, 1602, 1496 cm⁻¹

¹H NMR (400 MHz, D₂O) δ ppm: 7.57-7.52 (m, 4H), 7.48-7.39 (m, 5H), 5.23 (s, 1H), 3.83 (t, 0.1=4 Hz, 2H), 3.71-3.68 (m, 2H), 3.65-3.55 (m, 61-1), 3.47 (t, J=4 Hz, 2H), 3.4-3.3 (br s, 4H)

### Example II

### (±) 1-[(4-chlorophenyl)-phenyl-methyl]-4-[(3-methylphenyl)methyl]piperazine dihydrochloride (Meclizine dihydrochloride)

N-(4-Chlorobenzhydril) piperazine 0.35 mol) was taken in water (300 ml) and stirred at 25° C. Potassium carbonate (33.75 gm, 0.24 mol), tetrabutyl ammonium bromide (0.05 g) were added in sequence into it while stirring. 3-methyl benzyl chloride (59.0 gm, 0.42 mol) was then added into the reaction mixture while stirring. The reaction mixture was heated at 60° C. for 2 h. It was cooled to room temperature and extracted with ethyl acetate (100 ml). The ethyl acetate layer was washed with water. The ethyl acetate layer was concentrated to obtain Meclizine free base. Yield: Quantitative. Purity by HPLC: 98%. This was converted to hydrochloride salt by usual procedure.

IR (neat): 2391, 2289, 1490 cm⁻¹

¹HNMR (400 MHz, CDCl₃) δ ppm: 13.7 (s, 1H, HCl), 13.23 (s, 1H, HCl), 7.86 (s, 4H), 7.46-7.36 (m, 7H), 7.34-7.22 (m, 2H), 5.11 (s, 1H), 4.4-4.15 (m, 4H), 4.1-3.9 (m, 2H), 3.52-3.33 (m, 4H), 2.35 (s, 3H)

### Example III

### (±)2-[2-[4-[(4-Chlorophenyl)phenylmethyl]-1-piperazinyl]ethoxyacetic acid dihydrochloride (Cetrizine dihydrochloride)

N-(4-Chloro benzhydril) piperazine (10 gm, 34.9 mmol) was taken in water (30 ml) and stirred at 25° C. Potassium carbonate (3.37 gm, 24.4 mmol), tetrabutyl ammonium bromide (0.05 g) were added in sequence into it while stirring. 2-(2-chloroethoxy)acetamide (5.21 gm, 37.8 mmol) was then added into the reaction mixture while stirring. The reaction mixture was heated at 80° C. After the reaction is over (TLC), it was cooled to room temperature and extracted with toluene (20 ml). The toluene layer was washed with brine solution and dried. The organic layer was concentrated to obtain the 12.8 g compound. Yield: 95%. This was converted to cetirizine hydrochloride by hydrolysis of amide followed by formation of hydrochloride salt by usual procedure.

IR(neat):2983(br), 2389, 2291, 1739 cm⁻¹

¹HNMR (400 MHz, D₂O) δ ppm: 7.53 (d, J=8 Hz, 2H), 7.49-7.39 (complex m, 5H), 7.35-7.34 (m, 2H), 5.31 (s, 1H), 4.15 (s, 2H), 3.84 (t, J=4 Hz, 2H), 3.65 (br s, 4H), 3.46 (t, J=4 Hz, 2H), 3.42 (br s, 41-1)

### Example IV

### (±)2-{4-[(4-chlorophenyl)(phenylmethyl)piperazin-1-yl}ethanol dihydrochloride

N-(4-Chloro benzhydril) piperazine (10 gm, 34.9 mmol) was taken in water (30 ml) and stirred at 25° C. Potassium carbonate (9.65 gm, 69.9 mmol), tetrabutyl ammonium bromide (0.05 g) were added in sequence into it while stirring. Then 2-chloroethanol (4.1 gm, 50.9 mmol) was added into the reaction mixture while stirring. The reaction mixture was heated at 80° C. After the reaction is over (TLC), it was cooled to room temperature and extracted with ethyl acetate (20 ml). The ethyl acetate layer was washed with brine solution and dried. The organic layer was concentrated to obtain the 11 g compound. Yield: 94%. This was converted to corresponding hydrochloride salt by usual procedure

IR (neat): 3300, 2287, 1597, 1494, 1440 cm⁻¹

1H NMP (400 MHz, D2O) δ ppm: 7.53-7.32 (complex m, 9H), 5.27 (s, 1H), 3.85 (t, J=4 Hz, 2H), 3.59-3.52 (m, 4H), 3.39-3.34 (m, 6H).

### Example V

### (±)1-allyl-4-benzhydrylpiperazine dihydrochloride (Aligeron dihydrochloride)

Benzhydryl piperazine (10 gm, 39.6 mmol) was taken in water (30 ml) and stirred at 25° C. Potassium carbonate (10.92 gm, 79.3 mmol), tetrabutyl ammonium bromide (0.05 g) were added in sequence into it while stirring. Allyl bromide (7.18 gm, 59.5 mmol) was then added into the reaction mixture while stirring. The reaction mixture was stirred at 35-40° C. for 2 h. After the reaction is over (TLC), it was cooled to room temperature and extracted with dichloromethane (30 ml). The organic layer was washed with brine solution and dried. The organic layer was concentrated to obtain 6.5 gm of the desired compound and converted to corresponding hydrochloride salt by usual procedure.

IR (neat): 3051, 3041, 2954, 2401, 1496, 1456, 1433 cm⁻¹

¹HNMR (400 MHz, D₂O) δ ppm: 7.6-7.58 (m, 4H), 7.47-7.38 (m, 6H), 5.89-5.82 (m, 1H), 5.61-5.56 (m, 2H), 5.36 (s, 1H), 3.83 (d, J=7.2 Hz, 2H), 3.54-3.45 (m, 8H).

### Example VI

### (±)1-(4-tert-butylbenzyl)-4-[(4-chlorophenyl)(phenyl)methyl]piperazine dihydrochloride (Buclizine hydrochloride)

Benzhydryl piperazine (10 gm, 34.9 mmol) was taken in water (30 ml) and stirred at 25° C. Potassium carbonate (10.5 gm, 52.35 mmol), tetrabutyl ammonium bromide (0.1 g) were added in sequence into it while stirring. 4-tert-butyl benzyl chloride (9.5 gm, 52.3 mmol) was then added into the reaction mixture while stirring. The reaction mixture was heated to 90° C. for 3-h. After the reaction is over (TLC), it was cooled to room temperature and extracted with ethyl acetate (50 ml). The organic layer was washed with brine solution and dried. The organic layer was concentrated to obtain 13.95 g of the desired compound and converted to corresponding hydrochloride salt by usual procedure.

IR (neat): 2962, 2360, 1494, 1456, 1435 cm⁻¹

¹HNMR (400 MHz, CDCl₃) δ ppm: 13.79 (s, 1H), 13.19 (s, 1H), 7.85 (s, 4H), 7.54-7.52 (m, 2H), 7.45-7.37 (m, 7H), 5.08 (s, 1H), 4.22 (s, 4H), 4.06-3.99 (m, 2H), 3.48-3.37 (m, 4H), 1.28 (s, 9H)

We claim:

1. A novel process for the preparation of substituted diphenylmethyl piperazines of Formula I and pharmaceutically acceptable salts thereof 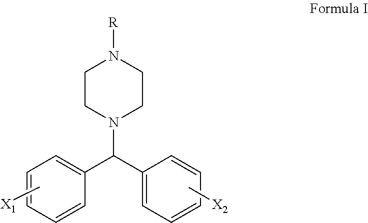
wherein X₁ and X₂ represent independently a hydrogen, a halogen, a straight or branched chain lower alkyl, alkoxy or a hydroxyl radical
and R is selected from groups such as acyl, alkyl, alkenyl, aralalkyl, aralalkenyl, hydroxyalkyl, aryloxyalkyl, alkoxyalkyl, aminoalkyl or its derivatives comprising,
reacting a compound of Formula II 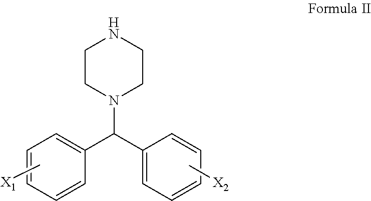
with a compound of Formula RX
wherein R is same as above
and X is suitable leaving group which includes halides, but not limiting use of other leaving groups such as tosylate, mesylate and activated acid groups using water as a solvent in presence of a catalyst and a base

2. The process as claimed in claim 1, wherein the reaction is carried out at temperature between 25 to 100° C. for 0.5 to 10 hours.

3. The process as claimed in claim 1, wherein the amount of compound of Formula RX used is in the range of 1 to 1.75 molar equivalents with respect to the compound of Formula II.

4. The process as claimed in claim 1, wherein the amount of water used is in the range of 2 to 5 volumes with respect to the compound of Formula II.

5. The process as claimed in claim 1, wherein the base used is selected from inorganic or organic base, the inorganic base is selected from alkali metal or alkaline earth metal carbonates or bicarbonates like potassium carbonate or sodium carbonate and the organic base is selected from triethylamine and the like.

6. The process as claimed in claim 1, wherein the catalyst used is selected from a phase transfer catalyst or an alkali metal halide, such that the phase transfer catalyst is selected from quaternary ‘onium’ salt of nitrogen or phosphorous, substituted with a residue namely alkyl or aralalkyl group.

7. The process as claimed in claim 1, wherein the catalyst used is selected from the group consisting of tetraalkylammonium halide, trialkylaryl ammonium halide.

8. The process as claimed in claim 1, wherein the amount of catalyst used is in an amount ranging from 0.1 to 1 wt % with respect to the compound of Formula II.

9. The process as claimed in claim 1, further comprising isolating the compound of Formula I, either by separation or by extraction, using an organic solvent selected from aromatic hydrocarbons, ethers, esters, halogenated hydrocarbons or alcohols.

10. The process as claimed in claim 1, wherein the compound of Formula I is optionally converted to its pharmaceutically acceptable salt.

11. The process as claimed in claim 1, wherein the compound of Formula I is racemic or optically active i.e. dextrorotatory or levorotatory.

12. The process as claimed in claim 1, wherein the compound of Formula I is selected from the group consisting of Cetirizine, Meclizine, Hydroxyzine, Buclizine, Aligeron or salts thereof.

13. A process as claimed in claim 1, wherein the compound of Formula I is 2-(4-((4-chlorophenyl)(phenyl)methyl)piperazin-1-yl)ethanol
